(19) United States
(12) Patent Application Publication
(10) Pub. No.: US 2023/0060377 A1
(43) Pub. Date: Mar. 2, 2023

(54) METHOD FOR CALCULATING A STIMULUS FOR THE CHANGE OF SHAPE OF A MEDICAL AID

(71) Applicant: K Line Europe GmbH, Düsseldorf (DE)

(72) Inventors: Ahmed Khaled El Banna, Kairo (EG); Sherif Kandil, Düsseldorf (DE)

(73) Assignee: K Line Europe GmbH, Düsseldorf (DE)

(21) Appl. No.: 17/642,291

(22) PCT Filed: Sep. 10, 2020
(86) PCT No.: PCT/EP2020/075409

§ 371 Date: Mar. 11, 2022

(30) Foreign Application Priority Data
Sep. 11, 2019 (DE) 10 2019 124 364.5

(51) Int. Cl. A61C 7/00; A61B 5/11; A61C 7/08; G05B 15/02

(52) U.S. Cl. CPC A61C 7/002; A61B 5/1114; A61C 7/08; G05B 15/02

(57) ABSTRACT

The invention relates to a method for calculating a stimulus for the change of shape of a medical aid in order to change the position of the medical aid from an actual position to a final target state. In order to achieve optimized treatment for the patient, a method comprising the following steps is provided:
Detecting the actual position of the body part to be corrected after a first correction step in a control unit,
Comparing the actual position of the body part to be corrected with a target position that is stored in the control unit,
If applicable, detecting, by the control unit, the deviation between the actual position and target position of the body part to be corrected,
Calculating with the control unit the stimulus that has to act on the medical aid to perform a second correction step, taking into account the deviation between the actual state and target state of the body part to be corrected, wherein the effect of the stimulus on the medical aid is saved in the control unit.

The invention further relates to a control unit.

### BACKGROUND OF THE INVENTION

The invention relates to a method for calculating a stimulus for the change of shape of a medical aid and to a control unit.

Medical aids comprise numerous products, all of which can be used to reduce or heal physical or organic defects or illnesses. They are aids that are used in or on the body or in conjunction with other medical aids such as braces, in particular tooth correcting devices, orthotics, extenders, fixators, implants or the like. The medical aids typically consist of a polymer or a combination of polymers or of a metal, or a combination or alloy of metals, that change shape in a precalculated manner under the influence of a stimulus, or they comprise such material. Many medical treatments require an incremental change of bone or tooth position, organs, or tissues. This incremental change must take place precisely.

Tooth correcting devices that change shape and therefore the position of a tooth from an actual position to a final target position are known, for example, from WO 2016/193424 A1 and WO 2017/079157 A1. These tooth correcting devices (also referred to in the following as “correcting devices” for short) typically consist of a polymer or a combination of polymers or of a metal or a combination or alloy of metals that change shape in a precalculated manner under the influence of a stimulus. An incremental correction of the tooth that requires a sequence of individual changes of shape of the medical aid mostly takes place. For other medical aids which are used, for example, in the correction of broken bones, bone lengthening or wound healing, incremental treatments are also known.

According to the current prior art, again, for example, in the case of a tooth correction, starting from the original tooth position, a medical aid, here a tooth correcting device, is designed that undergoes a first change of shape under the influence of a first stimulus and then effects a first change of the tooth position in a first correction step. A second correction step often follows, in which an additional stimulus acts on the medical aid and triggers a second change of shape. This process can be repeated. This procedure is used, for example, in correcting bone injuries, in bone lengthening, or in wound healing.

However, it has been found that the actual position of a tooth or bone to be corrected, or respectively a wound, often does not match with the expected target position after the first correction step, whether a tooth, a bone, or a wound turns out to be more or less mobile than assumed or the shape of the medical aid cannot be changed as previously calculated.

### SUMMARY OF THE INVENTION

It is therefore an object of the invention to achieve an optimized treatment for the patient.

This object is solved with a method as well as with a control unit both as disclosed herein.

The method according to the invention for calculating a stimulus for the change of shape of a medical aid in order to change the position of a medical aid from an actual position to a final target state has the following steps:
Detecting the actual position of the body part to be corrected after a first correction step,
Entering the actual position of the body part to be corrected into a control unit,
Comparing the actual position of the body part to be corrected with a target position that is stored in the control unit,
If applicable, detecting, by the control unit, the deviation between the actual position and target position of the body part to be corrected,
Calculating with the control unit the stimulus that has to act on the medical aid to perform a second correction step, taking into account the deviation between the actual position and target position of the body part to be corrected, wherein the effect of the stimulus on the medical aid is saved in the control unit.

Body parts that require a correction can be a tooth, a bone, an organ, or tissue. The medical aid can be a brace, in particular a tooth correcting device, an orthotic, an extender, for example, to bend or to lengthen bones, a fixator, for example, to align bones, an implant, surgical suture material or bandage material or the like, or a part of such a medical aid. The correction of the position of a body part can last for weeks, months, sometimes even years, as it is known, for example, for tooth corrections. A treatment plan from the initial position of the, for example, tooth or bone to be corrected to the final target position is usually divided into individual treatment steps that follow one after the other as correction steps. Each correction step begins with a change of shape of the medical aid. For this purpose, it is necessary to calculate a stimulus that brings about the desired change of shape. The treatment steps comprise an indicator of the time intervals at which changes of shape are to be made for the next correction step. Whenever reference is made to a tooth or bone in the context of this invention, all mentions also apply to the correction of the tooth position of multiple teeth or respectively of jaw malpositions or bones.

Detecting the actual position after a first correction step can take place conventionally in an analog manner, for example with an impression; preferably, however, the actual position is detected by digital measurement, for example by means of digital photography. Alternatively, however, the actual position can also be detected by a sensor that reports the current actual position of the tooth to be corrected or teeth to be corrected. In an advantageous embodiment of the invention, the sensor can be arranged on the medical aid, but the sensor can also be arranged on a scanner. For tooth corrections, an intraoral scanner is preferably used. The analogically or digitally detected actual position is then entered into a control unit. The input can be done manually by entering the data into a control unit. If the data is present in digital form, the input can be carried out directly and in an automated manner, for example, by initializing and carrying out the data comparison at the control unit. Advantageously, the input takes place at an input portal, in particular an input portal of the manufacturer of the medical aid or an input portal of the attending physician, for example a screen or a reader that can record data for a three-dimensional position description of a tooth. The input portal transmits the inputs to the control unit. The input portal can be displayed on a computer, it can be displayed by means of application software (app) on a smartphone, but it can also be specially configured as a separate device with the sole purpose of making inputs about the actual position.

Optionally, patient data can be entered into the input portal in order to incorporate the well-being of the patient into the optimization of the correcting medical treatment by means of the medical aid. This data can relate to the patient's wearing behavior with regard to the medical aid, behavior patterns of the use of the medical aid by the patient, his willingness to follow the treatment rules, the treatment progress, but also a plurality of inputs relating to vital signs, blood test results, information about saliva composition and quality and/or quantity, hormone status, or other analysis data, for example regarding excretions of the patient.

Preferably, the data is input in the way that can be easily processed by an algorithm or software, for example in assessment levels such as “low”-“medium”-“high” or in an assessment according to ratings, i.e., 1 to 5. It is preferred that the input portal displays the assessment levels for these inputs, so that the patient or the attending physician can quickly and easily select and input. Data that is detected, for example, by sensors, for example relating to the time periods in which the patient wears or uses the medical aid or relating to the manner in which the patient wears or uses the medical aid, can also be captured.

The control unit is designed to process software or algorithms. For simplification, calculations that take place by means of software, or respectively the processing of algorithms in the control unit, are described below as activity or actuation of the control unit.

A target position for the medical aid for each individual correction step of the treatment up to the final target position is saved in the control unit. After the actual position after the first correction step is entered, the control unit compares the actual position with the target position of the body part to be corrected. Any deviation that is found between the actual position and the target position is captured by the control unit after the input.

On the basis of the comparison of the actual position with the target position, the stimulus is calculated that is necessary to cause the medical aid to change shape in the manner necessary for a second correction step. Optionally, it is calculated which sections of the medical aid must be subjected to a stimulus in order to achieve a change of shape. In doing so, any deviation that is found between the actual position from the target position is taken into account: If, for example, the tooth or bone to be corrected moves farther than originally intended or if the patient cannot tolerate the rapid movement of the tooth or bone, the stimulus for the second correction step can be weaker or a different stimulus with a weaker effect can be selected because only a smaller correction movement is necessary. If the tooth or bone to be corrected moves less than originally intended or the patient signals that he desires a faster movement of the tooth or bone, a stronger stimulus or a different stimulus with a stronger effect can be used. The same applies to the treatment of scars or the treatment of burns or skin corrections. Optionally, this means that the well-being of the patient or the inputs regarding the condition of the patient are taken into account in the calculation of the stimulus for the next correction step. The time periods during which the patient wears the medical aid can also be incorporated into the calculation of the stimulus. Alternatively, a change of shape of the medical aid can also be brought about in each case by a combination of two or more stimuli. In this manner, the correction of the body part (tooth, bone, skin, tissue, organ) can be achieved efficiently, meaning quickly and with low stress on the patient.

The tooth correcting device described here by way of example can be a brace that is placed onto one or more teeth. It can also be configured as a wire that is placed, for example by means of holders, onto the tooth or teeth to be corrected. In general, the medical aid can be produced from any material. Preferably, it comprises exclusively dynamic synthetic material. However, it can also advantageously be a medical aid that consists of a combination of dynamic synthetic material and conventional, static synthetic material, wherein the shape of the static synthetic material cannot be changed by a stimulus (with the exception of mechanical force). Furthermore, synthetic material, meaning dynamic material or dynamic material in combination with static plastic material, can be used with another material, typically, for example, a plastic/metal combination. According to another alternative, the synthetic material can be combined with natural or synthetic rubber. Preferably, all the materials used contribute to aligning the tooth or bone, or respectively tissue or organ, i.e., transferring it to the final target position. The method according to the invention is carried out with a medical aid that is produced using a dynamic synthetic material that changes shape in a calculable manner under the influence of a stimulus. The change of shape of the medical aid effects a movement of the body part to be corrected from an actual position to a target position.

Such a material, typically a thermoplastic material, consists, for example, of two different polymers that abut one another, preferably abut one another in layers. In this case, the first polymer does not react to movement-inducing stimuli, while the second polymer reacts to movement-inducing stimulation, in particular by a shape and/or volume change. Movement-inducing stimuli can be contact with moisture or liquid, in particular water and water-containing liquids, temperature, in particular heat and/or radiation. Such plastics are also referred to as shape memory polymers (SMPs). Alternatively, metals or shape memory alloys (SMAs) can also be used for the medical aid.

Many stimulations act as a stimulus, each of which are tailored to the material properties of the tooth correcting device, to effect a desired deformation. A stimulus is selected in particular from the group comprising the parameters time, temperature, pH value, current or respectively electrical voltage, radiation, in particular infrared radiation, sound, illumination and liquid, e.g., water or solvent. Alternatively, the stimulus is selected from a combination of these parameters, for example, temperature and pH value, water and temperature, or time and temperature. However, a stimulus can also be mechanical force which brings about a change of shape of the medical aid.

If the composition of the dynamic synthetic material or alternatively the amount of the second polymer and its absorption capacity for a stimulus is known, the magnitude of the form change of the synthetic material generated by the change of shape or volume can be calculated. The same applies to SMA.

Such thermoplastic material is offered, for example, by the company Stratasys Inc. in conjunction with the 4D Printing Project, along with information about which form change the contact with a stimulus or with various stimuli effects. Stratasys Inc. hereby implements a printing method in practice that was developed by Skylar Tibbits of the Self-Assembly Lab at Massachusetts Institute of Technology MIT, Boston, USA, and for which Autodesk Inc. has developed the software with which synthetic material, in particular dynamic synthetic material, can be built by means of a 3D printer to form a product that changes its outer shape in a predictable manner under the influence of a movement-inducing and thus form-changing stimulus.

The subsequent deformability of the dynamic synthetic material or an SMA that is induced by external stimuli can be used by producing medical aid at least partially out of SMP or SMA, which change shape in a precalculated manner when acted upon by a stimulation. If the medical aid is a tooth correcting device, it comprises a wall that abuts the surface of the tooth or teeth to be corrected. Due to the deformation, the wall of the tooth correcting device, which deforms in a calculated manner, exerts force on the tooth which the wall abuts and moves the tooth as a result into the target position. In the same manner, the dynamic synthetic material or the SMA can be used as a component of a fixator, an orthotic, an extender or an implant when these medical aids are used in a treatment plan for which is it is a prerequisite that the medical aids undergo a change of shape at every correction step, in order, for example, to align or lengthen bones or to treat, in particular stretch, wounds or scars, but also to adjust implants to changing conditions as part of a treatment plan. The advantage of the method according to the invention is that a new medical aid does not have to be created for each step, and the change of shape takes place in an optimal manner, taking into account the physical development of the patient.

The information about the effect of the stimulus, meaning of the various parameters mentioned above, on the respective material of the medical aid are each made known by the manufacturer and are thus known when producing the medical aid. This information is saved in the control unit and is incorporated into the software, or respectively the algorithm, that is used to calculate the stimulus necessary for the second correction step.

The calculated stimulus is then output by the control unit and afterwards advantageously transmitted to a treatment device. The treatment device can be a device into which the medical aid is inserted or that is placed onto or connected to the medical aid. The treatment device is therefore, for example, configured as a container which—depending on the embodiment—can be tempered and/or filled with liquid and/or into which the radiation, for example, infrared radiation, can be introduced. The treatment device can also have a connector for connecting to the medical aid and introduce a stimulus, in particular current or heat, into the medical aid via the connection. The treatment device is optionally designed to subject parts of the medical aid specified by the storage unit to a stimulus. The treatment device can also be designed to apply mechanical force in order to bring about a change of shape of the medical aid, for example by bending or pressing individual sections of the medical aid. However, the treatment device can alternatively be integrated into the medical aid in order, for example, to use heat as a stimulus. Preferably, the treatment device is designed to apply two or more stimuli in combination. The treatment device is characterized in that it triggers the stimulus according to the specification of the control unit, which stimulus effects a change of shape of the medical aid. The stimulus usually acts on the medical aid for a specified duration. The change of shape of the medical aid thus takes place in sections until a maximum change of shape of the medical aid has been achieved. This process of shape change can typically be repeated as often as the medical aid can still be deformed by a stimulus, or until the final target position of the body part to be corrected is reached.

According to the invention, the control unit comprises a storage device (tracking system) for the temporal progression of the correction steps, in particular for the progression of the actual position of the body part to be corrected.

According to a preferred development of the method according to the invention, the temporal progression, in particular the entire temporal progression of the actual position of the body part to be corrected, is taken into account by the control unit during the calculation of the stimulus for the second correction step.

According to an advantageous development of the method according to the invention, the control unit outputs the duration of the second correction step, the size of the next stimulus, and/or the parameters selected or calculated for the stimulation via an output portal. The output portal can be configured in the same way as the input portal. Optionally, the storage unit stores this information and uses it if applicable for the calculation of stimuli for later correction steps.

According to a development of the method according to the invention, the duration of the second correction step output by the control unit, the size of the next stimulus, and/or the parameters selected for the stimulation are transferred manually to a treatment device. Alternatively, the control unit transmits the output duration of the second correction step, the size of the next stimulus, and/or the parameters selected for the stimulation to a treatment device automatically to carry out the next correction step.

Further optionally, the output portal can transmit information about the treatment progress to the patient, for example, the progress of the treatment can be displayed or warnings can be output, for example, if the medical aid is not worn for long enough. In addition, indicators of the time until the next correction step can be displayed. Furthermore, an indicator can be displayed to the patient regarding in which position the medical aid is to be inserted into the treatment device (for example, longitudinally/transversely or vertically/horizontally), or in which position the treatment device is to be arranged to the medical aid. In conjunction with the input portal, the output portal can also support a chat function that enables direct communication with the physician or manufacturer.

The method according to the invention preferably comprises a separate data set for each medical aid, which can be edited after entering an editing authorization in the input portal, for example, by entering a new actual position of the body part to be corrected. This ensures that, in each case, data is input about the associated medical aid, for example about a tooth correcting device, a fixator, an extender, or an orthotic.

The invention further comprises a control unit having means for calculating a stimulus and a storage device for data. The means for calculating are designed to calculate and, if applicable, select the stimulus for the next correction step, wherein data about the effect of at least one stimulus, preferably, however, about multiple stimuli, and data about the target position of the body part to be corrected are contained in the storage device for the starting position and for each individual correction step, and wherein data about the actual position of the body part to be corrected is entered in advance for the calculation of the stimulus via an input portal connected to the control unit. The means for calculating comprise software that calculates the stimulus for the second correction step from the target position and the actual position of the body part to be corrected after a first correction step and, if applicable, a deviation between the target position and the current actual position and the effect of the respective at least one stimulus on the medical aid. In doing so, the software can switch or choose the stimulus or a combination of stimuli for the second correction step, depending on the magnitude of the change of shape of the medical aid to be effected in the second correction step to move the body part to be corrected. In addition, the software can determine the intensity of the stimulus, meaning, for example, the level of the temperature, the duration over which the stimulus must act. The stimulus can be chosen depending on a chosen treatment device so that it is ensured that the treatment device can also apply the calculated stimulus to the medical aid.

The calculated stimulus for the second correction step is then emitted. The stimulus is either printed or displayed on an output portal, for example, a screen. Alternatively, the calculated stimulus is transmitted to a treatment device and implemented there, i.e., the stimulus acts on the medical aid.

Entering the current actual position of the body part to be corrected takes place as described above via an input portal. Advantageously, the input portal is connected to the control unit; further advantageously, the input portal also serves as an output portal.

It is further preferred if the input portal can only be opened for an input with an access authorization. It is further preferred if an input for a specific correcting device can only take place if the specific data set for this correcting device is opened by means of a code. This ensures that the upcoming change of shape of the medical aid takes place correctly, because erroneous incorrect operation is precluded after the access code has been input.

It is further preferred that the control unit and, if applicable, the treatment device are preset for the medical aid such that, if parts of the control unit or software are missing or fail, the activation of the medical aid by the stimulus takes place for a following correction step according to the original treatment plan, so that the treatment is not interrupted but can merely be resumed in a less optimized manner.

### BRIEF DESCRIPTION OF THE DRAWINGS

Details of the invention are explained below with regard to an exemplary embodiment. In the figures:

FIG. 1 shows a process diagram of the schematic mapping of the input portal and treatment device;

FIG. 2a shows a process diagram of the complete calculation of the stimulus;

FIG. 2b shows a process diagram of the calculation of the difference between the actual position and target position of the tooth to be corrected; and

FIG. 3 shows carrying out the change of shape.

### DETAILED DESCRIPTION

The following exemplary embodiment refers to a tooth to be corrected. However, it can also be applied to a bone to be corrected (aligning a broken bone, lengthening a bone) or to the correction of a scar or a wound. In a first step of the method according to the invention for calculating a stimulus for triggering a deformation of a medical aid, the actual position of the tooth to be corrected is detected and the final target position of the tooth to be corrected is detected. A treatment plan is created in which the path from the current actual position to the final target position is divided into at least two correction steps, often also multiple correction steps. Corresponding to the treatment plan, the tooth correcting device, in the following “correcting device” for short, is created. The material of the medical aid changes its shape in a precalculated manner under the influence of a stimulus, preferably from an actual position to a target position. The type and intensity of the stimulus influence the magnitude of the change of shape.

The necessary data of the actual position and the final target position of the tooth to be corrected, the target positions according to the individual correction steps, and the type and intensity of at least one stimulus, preferably multiple stimuli, are saved in a control unit. The control unit comprises a storage device for this purpose. The control unit is further equipped with a processor and with software that, after completion of a first correction step, calculates a stimulus for triggering a change of shape of the medical aid for the second or respectively next following correction step.

The control unit can be a separate device with an input portal, for example, a keyboard or a touchscreen mask, and advantageously with an output portal, for example, a screen display or a printer.

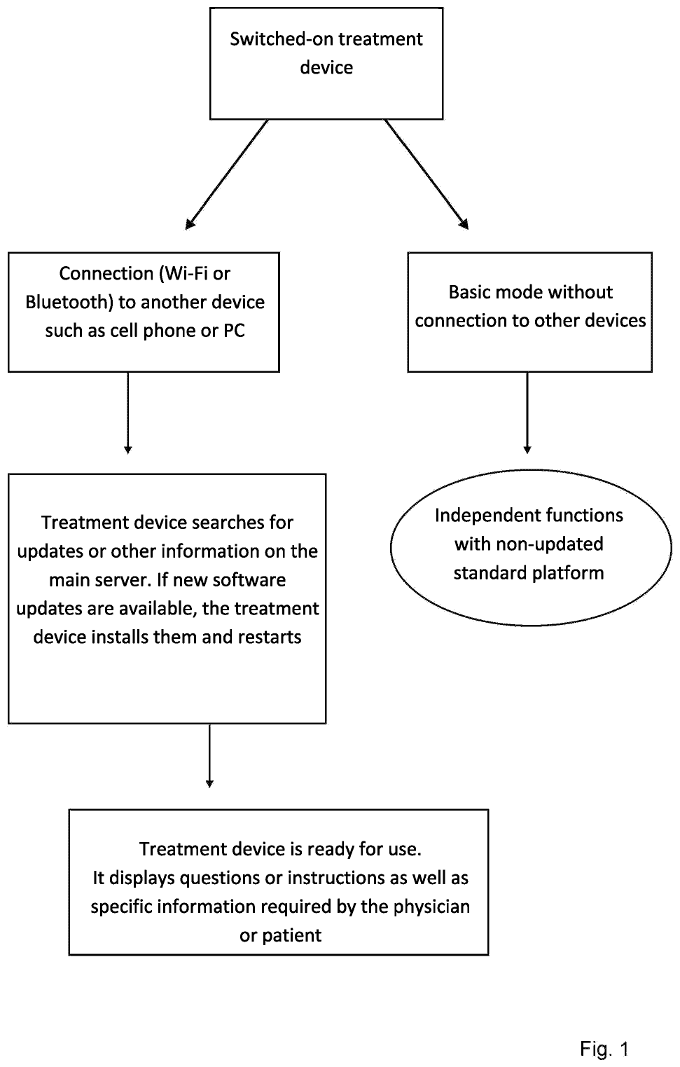

Preferably, the control unit is connected—as shown in FIG. 1—to a computer, for example, in the form of a smartphone, a tablet, or a desktop computer. To make the connection to the control unit, a connection to the input portal, for example as a separate application (app), can be downloaded, the input interface of which is connected to the input portal and the output interface of which is connected to the output portal of the control unit. The keyboard of the computer and the screen of the computer can be used for inputs into the input interface and thus into the input portal. Connecting the control unit to a computer has the advantage that the storage device and the software of the control unit can be kept constantly up to date through updates. Alternatively, a stand-alone control unit can be used.

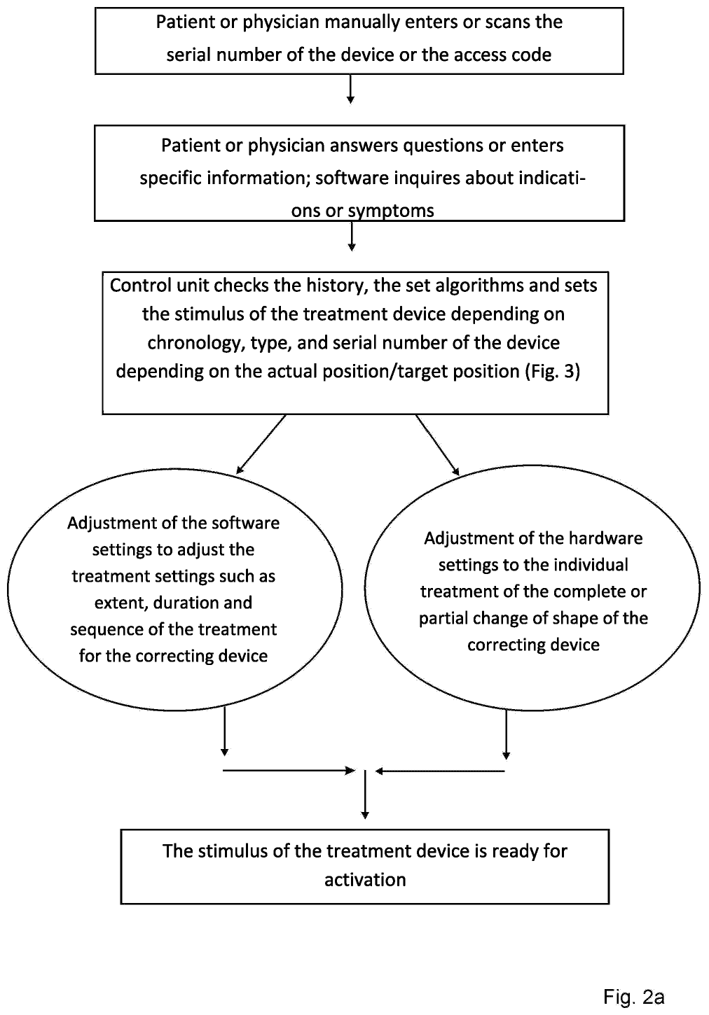

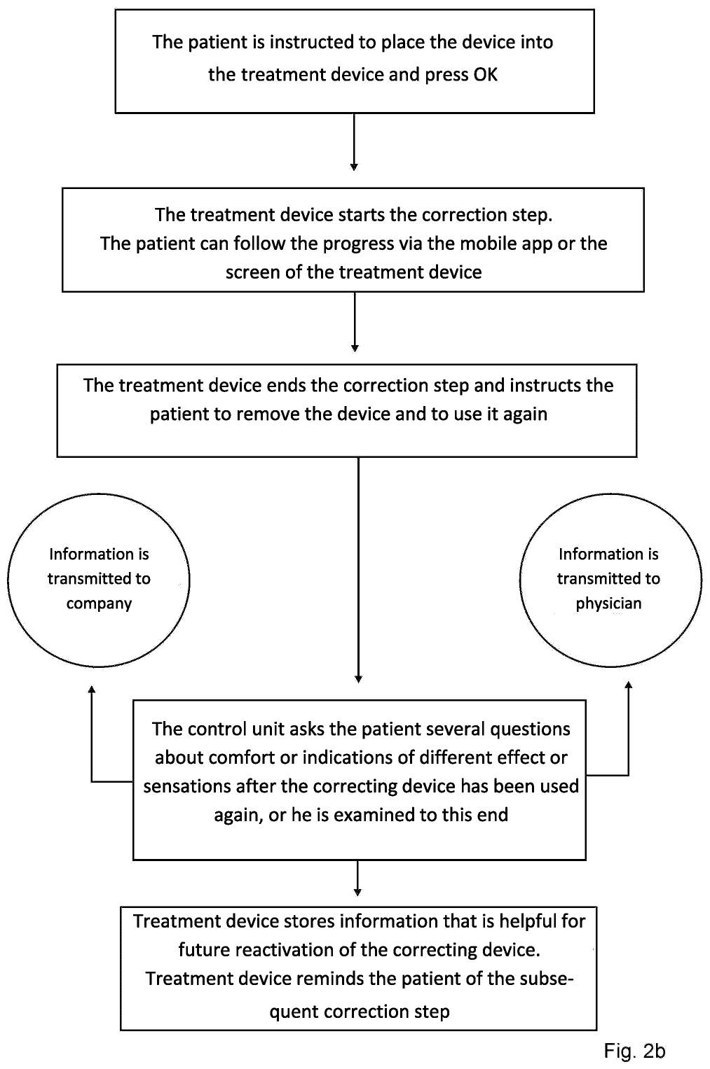

Starting from a control unit connected to a computer, after switching on and loading any available updates, this control unit is ready for data input, either for creating a new file for a new correcting device or for the evaluation of a correction step that has taken place and the calculation of a stimulus for a following correction step. The control unit or respectively the input portal can be designed for use for the patient, the attending physician, or the manufacturer of the medical aid, depending on who creates a new file for a medical aid or evaluates a correction step and calculates the stimulus for a further correction step. Advantageously, according to FIG. 2a, an access authorization is provided for each user of the control unit to ensure that only authorized persons and, if applicable, users trained to use the control unit make inputs. It is also recommended to create a separate file with a separate access code for each correcting device to ensure that a user who, for example, is overseeing multiple correcting devices as the attending physician always makes inputs for the correct correcting device.

At the end of a first correction step, the change of shape of the tooth or teeth to be corrected that has been achieved so far is evaluated. The currently achieved actual position of the tooth to be corrected is detected, either manually, for example, through an impression and measuring the impression, or digitally by measuring and determining the actual position of the tooth to be corrected. The data obtained in this way are entered into the input portal of the control unit, either via a keyboard or by means of data transmission, for example, via a cable, Bluetooth, or a wireless network connection (WLAN, Wi-Fi). Optionally, the input portal also asks the patient questions, for example, about the tolerability of the medical aid or the intensity of the correction. Advantageously, standardized responses (for example: good—moderate—poor; high—medium—low) are provided which can be processed in an algorithm that evaluates the first correction step.

To calculate the stimulus that the change of shape of the medical aid should effect for the following correction step, it is ascertained in accordance with FIG. 2b whether a deviation exists between the current actual position and the target position of the tooth to be corrected that is provided or expected according to the treatment plan. Such a deviation is ascertained, for example, by means of measuring the tooth position with an intraoral scanner or by comparing digital photographs; it can, however, also take place by creating a current impression and comparing it to the previous impression that was taken before beginning the completed correction step.

If such a deviation exists, this is taken into account for the calculation of the stimulus. Furthermore, in accordance with FIG. 2a, the responses to the questions of the control unit (if such questions have been asked) are taken into account for the calculation of the stimulus. The control unit or respectively the algorithm also takes into account the settings specified in the original treatment plan for the stimulus that should act on the medical aid at the beginning of the following correction step. The specified settings are now recalculated, taking into account any deviations of the actual position from the target position as well as the patients' responses, for example, about tolerability or intensity of the treatment, in order to move the tooth to be corrected into the final target position as quickly as possible with minimal stress to the patient. If applicable, the control unit or respectively the algorithm chooses a different stimulus and/or a different intensity, for example, a switch from heat to liquid or to a combination of stimuli, for example heat and liquid, depending on the extent to which a change of shape of the medical aid is necessary. The method according to the invention thus ensures that correcting the tooth position does not take place according to a rigid treatment plan specified at the beginning of the treatment, but rather that the correction is carried out in an optimized manner, taking into account the actual course of the correction. This results in a short correcting treatment that is gentle for the patient.

The recalculation of the stimulus for the change of shape of the medical aid is then emitted by the control unit via the output portal. According to a first alternative, the calculation of the stimulus is, for example, printed or displayed by the output portal and is manually transferred to a treatment device. The treatment device can be arranged directly on the medical aid, for example, as a heat source. The treatment device is often configured as a container in which the medical aid can be accommodated. The treatment device is further designed to allow at least one stimulus to act on the medical aid; advantageously, two or more stimuli can act on the medical aid, for example, temperature, liquid and radiation, each individually or in combination.

According to an alternative embodiment, the control unit is connected to and controls the treatment device. The connection can take place by installation in the housing of the treatment device, but it can also be made by a wired or wireless connection, in that the control unit sends actuation signals to the treatment device.

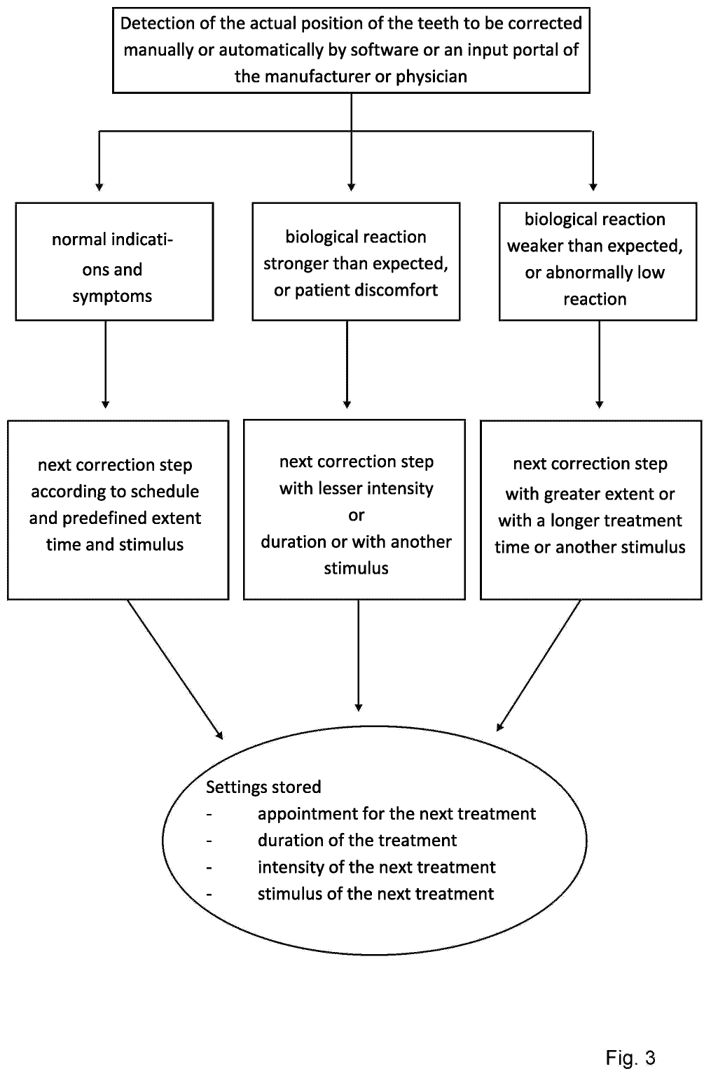

When controlling the treatment device in accordance with FIG. 3, the new calculation triggers the activation of one or more stimuli according to the specifications of the previous calculation of the control unit. If applicable, the control unit also causes the display of indicators in advance via the output portal, for example, the indicator to place the medical aid into the treatment apparatus, or the indicator to switch on the treatment apparatus after placing the medical aid and thus trigger the change of shape as a result of the action of the stimulus or the combined stimuli. Further optionally, the output portal displays the course of the action of the stimulus and the resulting change of shape of the medical aid.

After completion of the application of the stimulus, the output portal optionally displays information about the subsequent correction step, for example, how long the subsequent correction step takes, how long the medical aid is to be worn in each case in order to ensure optimal treatment success.

Optionally, after the medical aid is inserted by the patient, the output portal then shows the patient some questions that relate in particular to the fit of the correcting device, the shape of which has been changed, in the mouth as well as questions about the sensations (or paresthesia) while the medical aid is worn or questions about the effect of the medical aid on the teeth to be corrected or other teeth. In this manner, it is ensured that any erroneous deformations of the medical aid are discovered early.

As shown in FIG. 3, the control unit stores the data used to calculate the stimulus so that all information about the treatment is available at the end of the treatment. If applicable, this information is transmitted to the attending physician and/or the manufacturer of the medical aid. If the treatment with the tooth correcting device comprises more than two correction steps, all the information available from previous correction steps can be used to calculate the stimulus for a subsequent correction step.

Finally, the manner in which the differences between the actual position of the tooth to be corrected and the target position of the tooth to be corrected ascertained in accordance with FIG. 2b are incorporated into the calculation of the stimulus for the change of shape of the medical aid for the respective next correction step will be briefly explained. If it is found after the completion of a first correction step that no deviation between the actual position and the target position exists, the calculation of the stimulus for the change of shape of the medical aid takes place for the following correction step according to the original treatment plan.

If the tooth to be corrected has moved farther than expected or if the patient reports discomfort, then the treatment plan is changed for the next correction step. A less intense stimulus or a different, less intense stimulus is selected (for example, liquid instead of radiation) by the control unit or respectively by the algorithm.

If the tooth to be corrected has moved less than expected or if the patient explicitly reports good health, then the treatment plan is changed for the next correction step. A more intense stimulus or a different, more intense stimulus is selected (for example, a longer action of the stimulus) by the control unit or respectively by the algorithm. A combination of stimuli can also be used, always within the scope of the stimuli that the respective treatment device can cause to act on the medical aid.

It will be explained below how the control unit or respectively the software required to implement the method according to the invention is created. For example, ATMEL chips can be provided with an access code that limit access to the control unit to authorized persons, since the chip can no longer be opened without the correct control code. The access code can be created, for example, with AVR Studio software. As the control unit, for example, an Espressif Systems Wi-Fi/BLESOC microchip (for example, esp8266 WIFI/esp32 WIFI/BLE) can be used that is based on a 32-bit microcontroller that has a sufficient number of digital I/O access points to control all electronic peripheral devices and analog inputs for detecting sensor signals and that also has sufficient storage capacity to store the software codes.

To create the software or respectively the algorithm, an Arduino IDE platform can be used, that is suitable for creating interactive software. The software is then transferred to a chip by means of a suitable printer. Later, series-produced printed circuit boards (PCBs) can be used, on which the software is stored and which serve as the control unit. If the control unit is installed in a treatment device, this can advantageously connect to a server on its own, for example, of the manufacturer and download updates. It is preferred that an input screen connected to the input portal and, if applicable, a display about the progress or status of the change of shape can be downloaded onto external computers such as smartphones, tablets, or desktop computers as an application (app).

The chip or respectively the circuit board on which the software is stored can now control numerous processes, including Wi-Fi or Bluetooth connections for transferring data from computers such as smartphones, tablets, or desktop computers, the input and the output portals, which, if applicable, are displayed on these computers, the management of storage devices on which data about the patient and/or about the correcting device or treatment plan is stored, and, if applicable, also the treatment device, by which a stimulus acts on the medical aid to prepare a following correction step. In this case, the chip or respectively the circuit board controls, for example, the switching on and off of the respective stimulus, for example, a heating, a cooling, a radiation source or the addition of liquid, both individually and in combination. Thus, for example, a change of shape of a medical aid can require that first a specified amount of liquid, e.g., water, is let into a container containing the medical aid. It must be indicated beforehand that the medical aid has been correctly inserted into the container. This can be reported optically by a camera or by sensors or switches to the chip or the circuit board. Then, the water must first be heated and then cooled, wherein time specifications for the heating and the cooling must be complied with. Finally, it must be indicated that the change of shape is complete. Then, the data about the change of shape, both the information about the actual and the target positions as well as the inputs of the patient and the calculation of the stimulus, must optionally be stored and, if applicable, transmitted to the physician and manufacturer of the medical aid.

The control unit or respectively the chip or the circuit board can be brought into connection with a server of the physician or manufacturer, which server can be reached via a static IP address with a domain name. The control unit can retrieve updates or also modifications of the treatment device from the server. A Raspberry Pi server, for example, can be used, on which the operating system Raspbian, an Apache server, and contents programmed in PHP for web applications are installed.

In a second exemplary embodiment we will explain a possible embodiment of the method according to the invention using the control unit according to the invention. A patient wears a first correcting device for a week. According to the treatment plan, the medical aid should go through two changes of shape, each after one week. After entering the access code, the control unit or respectively software according to the invention is designed to first ask the patient which correcting device he is wearing and since when he has been wearing the medical aid. After entering further data on the actual position of the tooth to be corrected and optionally patient data, the stimulus necessary for the upcoming change of shape of the medical aid is calculated. It is taken into account here that the stimulus for the second correction step, meaning at the beginning of the second week, is to be calculated. The correct stimulus is therefore calculated without error and the treatment device is controlled accordingly by the control unit. After the second week, the same procedure takes place, taking into account the updated timeline. The stimulus is calculated for the third correction step, meaning for the third week. If the patient erroneously attempts to trigger a change of shape of the medical aid between the individual correction steps or after the completion of the correcting treatment, it is provided according to the present embodiment of the invention that the control unit displays that no change of shape is currently provided, or that the shape of the medical aid is up to date. This prevents erroneously undesired changes of shape from being made to the medical aid.

According to another exemplary embodiment, a single or multi-part orthotic or a plastic bandage is produced as described above from a dynamic synthetic material or in sections from a dynamic synthetic material in conjunction with a static synthetic material by means of a 3D printer. The orthotic or plastic bandage are individually adjusted to a patient to correct the bone position. The bone position, for example, the position of the foot bones, should be corrected in accordance with a multi-step treatment plan, for example, to restore the arch of the foot or the position of the toes.

A treatment plan is created that provides multiple correction steps. At the end of the first treatment plan, the bone position is detected, for example, by X-ray or another imaging method or by measuring the foot and the bone position. When calculating the following correction step, the actual position of the foot bone or foot bones to be corrected is detected and taken into account when calculating the stimulus necessary to deform the orthotic or the plastic bandage in order to carry out the next correction step. In this exemplary embodiment, more of the previously mentioned, individual parameters related to the patient can of course also be incorporated into the calculation of the stimulus.

A final exemplary embodiment relates to the care of wounds that takes place using the medical aid. The medical aid is in this case configured, for example, as a staple or as suture material. The changes of shape of the staple or of the suture material taking place in accordance with a multi-step treatment plan help to close wounds such that physiological damage is minimized and at the same time the aesthetic aspect of a scar is optimized. During the deformation of the staple or of the suture material by a stimulus according to the method according to the invention described above, the staples and suture material do not always have to be re-set and their shape can be changed incrementally in very small steps by stimuli calculated according to the invention and thus they can be optimally adjusted to the medical requirements. The shape of the suture material or respectively of the staples can be changed, for example, by heat or by radiation, but also by current.

1. A method for calculating a stimulus for the change of shape of a medical aid in order to change the shape of the aid from an actual position to a final target state comprising the steps:
detecting the actual position of the body part to be corrected after a first correction step in a control unit,
comparing the actual position of the body part to be corrected with a target position that is stored in the control unit,
wherein the method further comprises the steps
if applicable, detecting, by the control unit, the deviation between the actual position and target position of the body part to be corrected,
calculating with the control unit the stimulus that has to act on the medical aid to perform a second correction step, taking into account the deviation between the actual state and target state of the body part to be corrected, wherein the effect of the stimulus on the medical aid is saved in the control unit.

2. The method according to claim 1, wherein the stimulus is selected from the group consisting of the parameters time, temperature, pH value, current, radiation, sound, illumination, liquid and combinations of these parameters.

3. The method according to claim 1, wherein the actual position of the body part to be corrected is detected with an impression, by digitally measuring the position of the body part, or by signals from a sensor that detects the position of the body part.

4. The method according to claim 1, wherein the actual position of the body part to be corrected takes place by manual input into the control unit or by an input portal of the manufacturer of the medical aid or by an input portal of the attending physician.

5. The method according to claim 1, wherein the control unit is connected to a treatment device, which is designed to cause the calculated stimulus to act on the medical aid.

6. The method according to claim 1, wherein the control unit stores the temporal progression of the actual state of the body part to be corrected.

7. The method according to claim 1, wherein the control unit takes into account the temporal progression of the actual states of the body part to be corrected when calculating the stimulus for the second correction step.

8. The method according to claim 1, wherein the control unit calculates, emits and stores the duration of the second correction step, the size of the next stimulus, and/or the parameters selected for the stimulation.

9. The method according to claim 1, wherein the duration of the second correction step emitted by the control unit, the size of the next stimulus, and/or the parameters selected for the stimulation are transferred manually to a treatment device or wherein the control unit automatically uses the emitted duration of the second correction step, the size of the next stimulus, and/or the parameters selected for the stimulation to control a treatment device.

10. The method according to claim 1, wherein a separate data set is created for each medical aid, which data set can be edited after entering an editing authorization.

11. A control unit for carrying out a method according to claim 1, connected to an input portal for entering the actual state of the body part to be corrected and with means for calculating a stimulus for the change of shape of a medical aid as well as with a storage device wherein the effect of the stimulus on the medical aid is saved in the control unit, wherein the storage device of control unit is designed to store the temporal progression of the correction steps.

12. The control unit according to claim 11, wherein the control unit and input portal are spatially separated, and wherein the data from the input portal are transferred to the control unit.

13. The control unit according to claim 11, wherein the input portal is opened by means of an access authorization.
